(19) United States
(12) Patent Application Publication
(10) Pub. No.: US 2011/0034756 A1
(43) Pub. Date: Feb. 10, 2011

(54) SLEEP INDUCEMENT APPARATUS

(76) Inventors: Jeremy Hacking, Clitheroe (GB); Nicholas Hacking, Clitheroe (GB)

Correspondence Address: ADAMS INTELLECTUAL PROPERTY LAW, Suite 2350 Charlotte Plaza, 201 South College Street, CHARLOTTE, NC, 28244, US

(21) Appl. No.: 12/936,445

(22) PCT Filed: Apr. 6, 2009
(86) PCT No.: PCT/GB09/50333

§ 371 Date: Oct. 5, 2010

(30) Foreign Application Priority Data
Apr. 7, 2008 (GB) 0806236.6

(51) Int. Cl. A61M 21/00

(52) U.S. Cl. 600/26

(57) ABSTRACT

A sleep inducement apparatus including an interface unit, a pair of headphones and an electrode strap. A sleep monitor cooperates with the electrode strap to detect the state of sleep of a subject through a heart rate variability analysis. The control unit cooperates with electrodes in the headphones to apply an electrical stimulation to the subject's nervous system in order to initiate a sleep condition. As the subject moves from a wakeful state towards a state of sleep, the monitor regulates the control unit such that, when the subject is asleep, the control unit does not supply electrical pulses.

The present invention relates to apparatus for inducing sleep and in particular, although not exclusively, to non-invasive, re-usable, and portable sleep inducement apparatus and a method thereof.

There are many sleep disorders that people (herein referred to as “subjects”) may suffer from and as a result experience difficulty getting to sleep. The sleep disorders may include chronic insomnia, transient insomnia (i.e. that brought on by stressful situations), or “jet-lag syndrome” which occurs due to the effects of long distance flying. Some sleep disorders may be resolved by treating the underlying problem or by improved “sleep hygiene”, which aims to improve sleeping practices. However, often subjects with sleep disorders, especially transient and jet-lag disorders, require additional help to go to sleep.

Subjects may choose to attempt to induce sleep via drug therapy. Such sleep medications (herein referred to as “sleeping pills”) are not intended for long term use and prolonged use can lead to addiction. Additionally, subjects using sleep medications to induce sleep may experience some or all of the following side effects: dizziness, lack of co-ordination, forgetfulness, constipation, urinary retention, blurred vision and dry mouth and throat. Subjects may also quickly develop a tolerance for the drug.

It is an object of the present invention to solve one of the above or other disadvantages.

It is a further aim of the present invention to provide sleep inducement apparatus with limited side effects and reduced accommodation (i.e. reduced effectiveness of the remedy due to the subject building immunity to repeated use).

It is a further aim of the present invention to provide a sleep inducement apparatus that may be used inconspicuously by the subject such that the apparatus may be used to induce sleep in public or semi-public situations, for example on board an aeroplane.

According to the present invention there is provided an apparatus and method as set forth in the appended claims. Other features of the invention will be apparent from the dependent claims, and the description which follows.

According to an exemplary embodiment there is provided an apparatus comprising: a sleep monitoring means that is arrangeable to detect the wakefulness of a subject; and a stimulus means; wherein in use, the sleep monitoring means regulates the stimulus means according to the state of wakefulness of the subject.

Preferably, the stimulus means provides a stimulus to the subject. The stimulus may comprise electrical stimulation.

In a first exemplary embodiment the apparatus comprises a sleep inducement apparatus. Here the stimulus means includes a control unit and at least one electrode that is contactable with a subject's skin. The control unit being arrangeable to provide electrical pulses to the or each electrode. The sleep monitoring means detects when the subject is awake. The sleep monitoring means being arrangeable to regulate the control means such that the control means provides the electrical pulses to the or each electrode when the subject is awake.

Preferably the sleep monitoring means is arrangeable to detect when the subject moves toward a state of sleep.

The sleep monitoring means may regulate the control unit such that when the subject moves toward a state of sleep, the electrical pulses reduce.

Preferably the sleep monitoring means is arrangeable to detect when the subject is asleep. The sleep monitoring means may regulate the control unit such that when the subject is asleep no electrical pulses are provided to the or each electrode. Advantageously, because the electrical pulses are not provided when the subject is asleep, the subject is less likely to develop accommodation to the apparatus.

Preferably the sleep monitoring means continuously monitor the subject. The sleep monitoring means may be arrangeable to detect the subject waking. The sleep inducement apparatus may include a time means. The time means may be programmable with a user defined waking time. The sleep monitoring means may regulate the control unit such that if the subject begins to wake before the waking time, the control unit provides electrical pulses to the or each electrode. The electrical pulses may be gradually re-introduced. The time means may comprise a clock.

Preferably the stimulus means comprises an audio means. The audio means may comprise a music player and headphones. The music player may be arranged to play music through the headphones. Advantageously, the music may cut out background noise to aid sleeping conditions. The sleep monitoring means may regulate the music player such that music plays when the subject is awake. The sleep monitoring means may regulate the music player such that music does not play when the subject is asleep. The time means may regulate the music player to sound an alarm when the waking time is reached.

Preferably the apparatus comprises an interface unit. The interface unit may house the music player and control unit.

Preferably the sleep monitoring means comprises a sleep monitor and a pair of monitoring electrodes. The pair of monitoring electrodes may be arrangeable to measure the electrical activity of the heart. The sleep monitor may process the parameters detected by the pair of monitoring electrodes in order to determine the state of wakefulness of a subject. The sleep monitor may determine the state of wakefulness of the subject by monitoring the heart rate variability interval.

Suitably, the pair of monitoring electrodes are arrangeable about a subject's chest or body. Here, the pair of monitoring electrodes maybe incorporated in a strap or into the apparatus or in self adhesive. The strap or apparatus or adhesive may be arrangeable about the subject's chest or body. Alternatively, the pair of monitoring electrodes may be incorporated into the headphones such that they contact the subject about the ear region. The pair of monitoring electrodes may be connectable to the interface unit by a first lead. The sleep monitor may be housed within the interface unit.

The sleep monitor and pair of monitoring electrodes may be connected via the first lead.

Preferably the headphones are connected to the interface unit by a second lead. The music player and headphones may be connected via the second lead.

Suitably the or each electrode are arranged on the headphones. At least one electrode may be arranged on each ear muff. The or each electrode may be electrically connected to the control unit via the second lead. Accordingly, the second lead may provide an electrical connection between the music player and headphones and a separate connection between the control unit and each electrode. The first and second leads may comprise at least partially the same lead.

Suitably, a common electrode may be provided with dual purpose as the or each electrode for providing the stimulus and also the pair of monitoring electrodes.

Preferably the interface unit is programmable. The interface unit may include a computer interface socket. A computer may be connectable to the interface unit via the computer interface socket in order to programme the interface unit. The interface unit may be programmable with the parameters of the control unit and/or the waking time and/or the music player.

According to an alternative exemplary embodiment the apparatus comprises a warning apparatus. The warning apparatus may be used to warn a subject when the subject reaches certain stages of wakefulness. The stimulus means may comprise an alarm. The sleep monitoring means may be as previously described. Accordingly the sleep monitoring means may regulate the stimulus means to sound an alarm when the subject moves towards a state of sleep. The stimulus means may also comprise a control unit and electrode as previously described. The sleep monitoring means may regulate the control means to supply electrical pulses to the electrodes in order to invoke a prickling sensation such that the subject moves towards a more wakeful state.

The present invention will now be described, by way of example, and with reference to the following drawings in which:

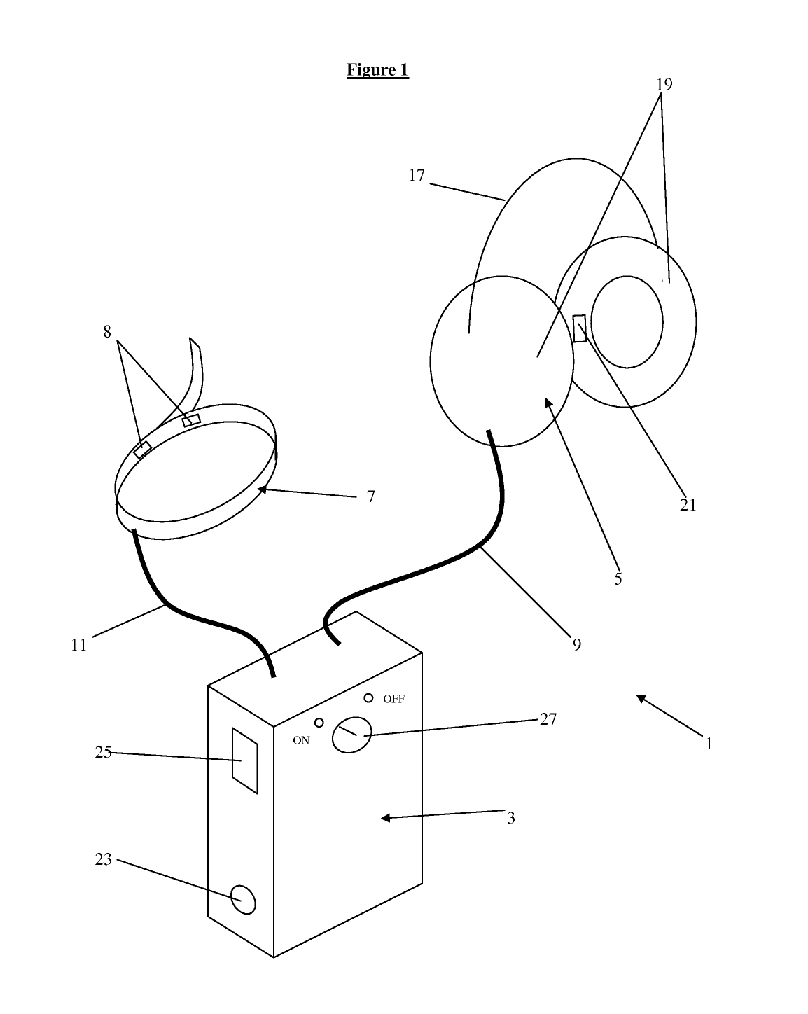

FIG. 1 shows an embodiment of the present invention.

Referring to FIG. 1, a sleep inducement apparatus 1 comprises an interface unit 3, a pair of headphones 5 and an electrode strap 7. The headphones 5 and electrode strap 7 are connected to the interface unit 3 by detachable leads 9 and 11 respectively. The interface unit 3 houses a sleep monitor, a music player and a control unit. The sleep monitor co-operates with the electrode strap to detect the state of sleep of a subject through a heart rate variability analysis. The music player co-operates with the headphones to play music therethrough. The control unit co-operates with the headphones 5 to apply a stimulus to the user. Here the stimulus is an electrical stimulation of the subject's nervous system in order to initiate a sleep condition.

In use, the subject attaches the interface unit 3 to their clothing, such as night-wear, and arranges the electrode strap 7 about their upper thorax (upper chest). If the subject is in a brightly lit or public place, a soft padded blindfold (not shown) may also be worn. The interface unit 3 is switched on and the subject attempts to sleep.

When switched on the sleep monitor analyses the state of wakefulness of the subject. When the user is in a state of wakefulness, white noise is played softly through the headphones and the control unit 5, emits a series of TENS like electrical pulses to the user's skin through the headphones.

As the subject moves from a wakeful state towards a state of sleep, the sleep monitor 3 regulates the control unit such that, when the user is asleep, the control unit does not supply electrical pulses to the user. Consequently, the subject is not submitted to electrical pulses during sleep, which may otherwise reduce the effectiveness of repeated use of the apparatus due to accommodation affects.

Once the subject is asleep, the sleep monitor continues to monitor the user for evidence of awakening in which case the white noise and electrical stimuli are re-introduced.

The various parts of the present invention will now be described in more detail.

The electrode strap 7 includes a pair of electrodes 8 that, in use, locate about the chest of the subject. The electrodes are electrically connected to the sleep monitor which includes electronics for recording the parameters of the electrical circuit thereformed. The electrodes thus provide a means of tracking the current flowing in the heart muscle as a result of the natural electrical activity of the heart. Such measurements, when graphed, are referred to in medicine as an electrocardiograph (ECG, also called EKG in certain localities). The measured potentials are plotted against time and the end result is the familiar shape described in medical and physiological literature as the “QRS” complex, which is normally preceded by a “P” wave and followed by an “R” wave.

The “R” wave of the QRS complex represents the electrical currents generated when the large chambers of the heart (the ventricles) contract in systole. The heart rate may therefore be defined by the number of R waves occurring in a given time. Furthermore, the time interval between consecutive R-waves is therefore inversely proportional to the heart rate. Analysis of a series of such time—intervals is termed R-R interval analysis.

The R-R interval can be expected to be under the control of a number of interacting feedback mechanisms. In healthy systems the R-R interval is typically not constant in time, but fluctuates about some nominal value, wherein the distribution of values over time is statistically normal (Gaussian) and the incidence of any particular value is essentially chaotic.

In contrast to the normal physiological state, when homeostasis functions optimally, the characteristic of the measured parameter changes in abnormal states. This is indicative of the fact that the normal control mechanisms are in an extreme state. As such the R-R intervals would not fill a normal distribution, rather it has been found that there would be a small set of discrete abnormal values which the measured rate switches between. Thus the loss of randomness in the R-R interval is indicative of biological stress.

The use of R-R interval analysis to detect the state of wakefulness is discussed in more detail in American Journal of Cardiol 1990 Feb 1;65(5):391-3. Reproducibility and circadian rhythm of heart rate variability in healthy subjects; Huikuri H V, Kessler K M, Terracall E, Castellanos A, Linnaluoto M K, Myerburg R J; Division of Cardiology, University of Miami Medical School, Florida; PMID:2301268 [PubMed-indexed for MEDLINE] the contents of which are hereby incorporated. This study shows that there is a detectable change in R-R variability (largely due to changes in autonomic functions) with the onset of sleep, with changes in the type of sleep, and with the onset of awakening from sleep.

The sleep monitor measures the R-R interval between the steep ascending slope of each R wave and analysis of the R-R variability. The music player and control unit are regulated by the sleep monitor according to the state of wakefulness of the subject.

The headphones 5 comprise a headband 17 and two ear muffs 10 and co-operate with the music player via the lead 9 to play music in any well-known manner. The headphones 5 also include transcutaneous electrodes 21. The electrodes 21 are positioned so as to contact the users' skin when the headphones are worn. The lead 9 also provides an electrical contact between the electrodes 21 and control unit.

The control unit and electrodes 21 may comprise a transcutaneous electrical nerve stimulator (TENS) unit. TENS units are widely used in the control and management of pain. TENS units comprise an electronic device which produces electrical signals to stimulate a subject's nerves via one or more (usually two) electrodes that are connected to a user's unbroken skin and either side of the area in which the pain occurs. A drawback to such pain relief is that it is widely accepted that the continuous use of TENS leads to it becoming ineffective; the wearer becomes refractory to the analgesic effect. This is believed to be due to neural adaptation. TENS machines for use in pain relief are discussed in more detail at reference “Transcutaneous Electrical Nerve Stimulation, Vibration and Acupuncture as Pain-Relieving Measures”; Hanson & Lundberg; in Textbook of Pain 4^{th} Edition; Editors Wall P. D. and Melzack R.; 00 1341-1351. However, a brief review of a TENS machine is as follows:

The electronic device delivers pulses of electrical energy to the electrodes. Generally, a TENS machine includes controls to vary the parameters of the electrical pulses. For instance the duration (or width) of each pulse may be varied from around 40 to 250 micro seconds, the pulse rate may be varied from around 1 pulse per second to 200 or 250 pulses per second, and the current intensity (strength) is typically variable between 0 to 100 mA. The subject may control the variables in order to find a setting that works best for each individual user.

There is experimental evidence to suggest that the TENS unit controls pain via more than one mechanism. At high pulse rates (90-130 Hz), it is thought that the unit stimulates low level nerves to carry the pulse signals to the brain. The spinal cord is only able to carry a certain number of signals. As such, the signals created by the TENS unit block pain signals carried by other nerves from reaching the brain. At low pulse rates (2-5 Hz) the unit stimulates nerves in order to initiate the subject's body into creating it's own natural pain relieving chemicals. It would appear that low frequency TENS is of particular value in nociceptive (i.e. non-neuropathic) pain and that the principle mechanism of action is medicated by the release of centrally-acting endorphins.

Endorphins have several physiological effects which have been compared to those of the exogenous opioids. Analgesia and somnolence are attributed to activation of the μ1 opioid receptor by either endogenous or exogenous ligands. Experimental evidence shows that blockage of the central U1 receptors inhibits the analgesic effects of both opioids and stimulation therapies (such as TENS and acupuncture).

It has been found that use of a TENS-like current at the low frequency (around 4 Hz) can advantageously aid the ability of a subject to reach a state of sleep. It is thought that this occurs by exploiting the central, predominantly endorphin-mediated, effects of peripheral stimulation by the TENS-like currents applied via the electrodes 21 to the users' skin.

The interface unit 3 comprises a small plastic housing for the electronics of the sleep monitor, music player, and control unit. The housing is contained within a thick sleeve of gel-padding. The housing also houses an internal rechargeable battery. The rechargeable battery powers the electronics for the sleep monitor, music player and control unit. The rechargeable battery is rechargeable by connecting a charging lead (not shown) to charging socket 23. When the charging lead is connected to the charging socket 23, re-charging occurs, and the electronics are isolated for safety.

The interface unit 3 also includes an interface connection, such as a USB port 25. The USB port 25 allows a computer or other programming machine to connect to the interface unit 3. As such, via the computer and appropriate software, the parameters of the control unit may be set. A set time for waking is also programmed into the interface unit via the computer.

The interface unit includes a power on/off switch 27. The external features of the interface unit will be low-profile to minimise discomfort, should the subject's body press upon the unit.

In use, the subject programs the sleep inducement apparatus 1 by connecting a computer to the USB port and setting the preferred parameters for the control unit, music/white noise, and a waking time. Alternatively, the existing parameters or default parameters may be used without requiring the step of programming apparatus. The sleep inducement apparatus is then worn with the electrode strap and the headphones arranged in place. The on/off switch on the interface unit 3 is then switched to on. The sleep monitor monitors the subject's R-R interval and ascertains that the user is awake. Accordingly, the music player and control unit are activated. The TENS-like pulses act to release endorphins in the subject which promote sleep. As the user begins to sleep, the sleep monitor detects a loss of variability in the R-R interval and accordingly controls the control unit so that when the subject is asleep, the TENS-like pulses are not applied. The music may also be stopped when the subject is asleep.

Accordingly, because the electrical stimulus is reduced to zero when the user is asleep, the user does not receive unnecessary exposure to the hypnotic signal (electrical pulses) and consequently the likelihood of the user developing a refractory state is reduced.

Once the user is asleep, the sleep monitor continues to monitor the subjects R-R variability, looking for evidence of awakening. If it is determined that the user is waking up more than one hour before the desired time, then the white noise and electrical TENS-like stimuli are gradually re-introduced.

When the waking time is reached, the music player may be triggered to play an alarm of steadily increasing intensity through the headphones and also small bursts of electrical stimulation (i.e. using the control unit and electrodes in the headphones) are used to provoke a pricking sensation in order to rouse the sleeper.

Accordingly, the sleep inducement apparatus provides a unit that can be used discreetly to promote sleep. Moreover, because the apparatus includes a sleep monitor, the accommodation affects of the apparatus are reduced, for instance, as compared to a TENS unit incorporating a timer set for a pre-determined time, which assumes that sleep will come within a pre-defined period and since the apparatus is required by subjects suffering insomnia; this is not an assumption that can be made. Moreover, the sleep monitor continues to monitor the wakefulness of the subject such that if the subject begins to wake, the apparatus operates again. As such the subject does not necessarily regain wakefulness.

In order to increase the un-obtrusive nature of the apparatus, the electrodes for measuring the subject's state of wakefulness may be arranged on the headphones. Moreover, it is preferred if the interface unit is also incorporated into the headphones.

It will be appreciated that the apparatus may be provided with a lead including standard TENS-electrodes so that the apparatus may be used as a TENS machines for pain relief.

Furthermore, the apparatus may be used as an apparatus for keeping a subject awake. For instance rather than supplying the music and electrical stimuli when the subject is awake, the apparatus may be used to sound an alarm or invoke the prickling sensation in order to wake the subject, should it be detected that the subject was falling asleep. This may be advantageous, for example, for train drivers or other users who need to stay awake.

The above application has been described wherein the sleep monitor determines the state of wakefulness of the subject by monitoring the subject's heart rate through an R-R interval analysis. However, it has been found that the cost and complexity of the apparatus can be reduced by employing an alternative heart rate monitor.

Specifically, it has been found surprisingly beneficial to use a pulse oximeter device.

Pulse oximeters are medical devices that indirectly measure the oxygen saturation of a subject's blood. This is done by emitting light through a translucent part of a subject's body (usually a fingertip or earlobe) and recording the received amount. Most modern pulse oximeters use a pair of LED's, one LED emitting a red wavelength (typically 660 nm) and the other an infrared wavelength (typically 905, or 940 nm). Because the absorption of the translucent part of the subject's body differs significantly between oxyhemoglobin blood and its deoxygenated form, the ratio of the absorption of the emitted light can be calculated. As is well-known, the oxygen saturation of a subject's blood can therefore be measured in a non invasive way.

Also because the monitored signal bounces in time with the heart beat due to the arterial blood vessels expanding and contracting with each heart beat, the heart beat can also be monitored. A state of wakefulness of the subject can then be monitored as herein described.

The pulse oximeter can be incorporated into the apparatus as a finger clip. However, preferably the headphones are adapted to include a clip for clasping a subject's earlobe. One side of the clip including light emitting elements and the other a detector as is widely known. Thus, both the electrical stimulation and equipment to monitor the heart rate are incorporated into the headphones. As, in this example, there is no need for the electrode strap, the apparatus simply comprises an interface unit and a pair of headphones connected thereto with a wire. Consequently, to use the apparatus a subject need only place the headphones over their ears ensuring the electrodes are in contact with skin, and arrange the earlobe clip about their lobe.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A sleep inducement apparatus for assisting a subject to reach a state of sleep, the apparatus comprising:
a sleep monitoring means that is arrangable to detect the wakefulness of a subject; and
a stimulus means that includes a control unit for generating Transcutaneous Electrical Nerve Stimulus (TENS) pulses and at least one electrode that is contactable with a subjects' skin for applying TENS pulses; wherein
the sleep monitoring means is arranged to regulate the control means, such that, in use, the control means provides electrical pulses to the or each electrode when the sleep monitoring means detects that a subject is awake.

2. The sleep inducement apparatus according to claim 1 wherein the sleep monitoring means is arranged, in use, to detect when the subject is asleep and regulates the control unit such that when the subject is asleep no electrical pulses are provided to the or each electrode.

3. The sleep inducement apparatus according to claim 1 wherein the sleep inducement apparatus includes a time means that is programmed with a waking time and wherein the sleep monitoring means is arranged, in use, to monitor the subject such that it detects the subject waking, and the sleep monitoring means regulates the control unit such that if the subject begins to wake before the waking time, the control unit provides electrical pulses to the or each electrode.

4. The sleep inducement apparatus according to claim 1 wherein the stimulus means includes a music player and headphones and the or each electrode are arranged on the headphones.

5. The sleep inducement apparatus according to claim 1 wherein the sleep monitoring means comprises a pulse oximeter monitor.

6. The sleep inducement apparatus of claim 5, according to claim 4, wherein the pulse oximeter comprises an earlobe clip and a sleep monitor, the earlobe chip clip being incorporated into the headphones.

7. The sleep inducement apparatus according to claim 1, wherein the sleep monitoring means comprises a sleep monitor and a pair of monitoring electrodes, which are arranged, in use, to measure the electrical activity of the heart such that by processing the parameters detected by the pair of monitoring electrodes the sleep monitor determines the state of wakefulness of a subject.

8. The sleep inducement apparatus according to claim 1, wherein the apparatus comprises an interface unit, and the interface unit houses any of the music player, control unit and sleep monitor.

9. The sleep inducement apparatus according to claim 8, wherein the interface unit is programmable with the parameters of the control unit or the waking time or the music player or any configuration thereof.

10. A method of inducing sleep comprising using a sleep inducement apparatus and arranging a sleep monitor of the apparatus on a subject wherein the sleep monitor detects the state of wakefulness of the subject, the apparatus further comprising a stimulus means that comprises a control unit and at least one electrode for applying a Transcutaneous Electrical Nerve Stimulus (TENS) pulses, the method further comprising causing the sleep monitor to regulate the control unit to provide electrical pulses to the or each electrode when the sleep monitor detects a subject to be in a state of wakefulness.

11. The method as claimed in claim 10 wherein the method comprises continuously monitoring the subject with the sleep monitor, such that when the sleep monitor detects that the subject is asleep, the sleep monitor regulates the control unit such that it does not provide electrical pulses to the or each electrode.

12. The method as claimed in claim 11, wherein the apparatus comprises a time means that is programmed with a waking time and the method comprises monitoring the subjects state of wakefulness and, should it be detected that the subject is awaking before the waking time, causing the sleep monitor to regulate the control means to provide electrical pulses to the or each electrode.
